(19) United States
(12) Patent Application Publication
(10) Pub. No.: US 2019/0201639 A1
(43) Pub. Date: Jul. 4, 2019

(54) LIQUID DISPENSER, IN PARTICULAR AN INHALER

(71) Applicant: APTAR RADOLFZELL, GMBH, Radolfzell (DE)

(72) Inventors: Magnus LUTZ, Uhldingen-Mühlhofen (DE); Pia MEINEL, Konstanz (DE)

(21) Appl. No.: 16/311,756

(22) PCT Filed: Jul. 21, 2017
(86) PCT No.: PCT/EP2017/068484

§ 371 Date: Dec. 20, 2018

(30) Foreign Application Priority Data
Jul. 27, 2016 (EP) 16181528.7

(51) Int. Cl. A61M 15/00; A61M 11/02; A61M 16/06; A61M 11/00; B65D 83/20; B65D 83/22

(52) U.S. Cl. CPC A61M 15/009; A61M 11/02; A61M 15/0016; B65D 83/226; A61M 16/06; A61M 11/007; B65D 83/205; A61M 15/0021

(57) ABSTRACT

A liquid dispenser for discharging cosmetic or pharmaceutical liquids, in particular in the form of an inhaler. This liquid dispenser includes a switchable valve mechanism, a base, and a discharge head which, for actuation of the valve mechanism, is movable with respect to the base in a superposed axial and rotational movement or in a rotational movement between an opening position, in which the valve mechanism is opened, and a closure position, in which the valve mechanism is closed. It is proposed that, in a delivery state of the liquid dispenser, a rotational movement of the discharge head with respect to the base is blocked by a tamper-evident portion that is separable from the dispenser.

### FIELD OF USE AND PRIOR ART

The invention relates to a liquid dispenser according to the preamble of claim 1.

A liquid dispenser of the type in question is configured as a portable liquid dispenser which the user or patient can usually carry around in a bag or a coat pocket. In particular, the liquid dispenser of the type in question can be configured as an inhaler and for this purpose discharges liquid in atomized form from a liquid reservoir of the liquid dispenser.

In addition to having a liquid reservoir, a liquid dispenser of the type in question has a discharge device comprising a base and a discharge head movable relative to the base. A switchable valve mechanism of the liquid dispenser can be transferred to an open or closed state by the relative movement.

The mobility of the discharge head relative to the base can be a purely rotational relative mobility. However, it is preferably a superposed axial and rotational relative mobility. In particular, in a dispenser of the type in question, provision can be made that the relative mobility corresponds to that of a thread.

### Problem and Solution

For a dispenser of the type in question, the problem addressed by the invention is to provide structurally simple means to prevent a situation where the liquid dispenser, prior to its first use, is accidentally activated by a rotation movement of the discharge head relative to the base, either during handling before the sale of the dispenser or by the end customer.

According to the invention, a liquid dispenser for discharging cosmetic or pharmaceutical liquids, in particular in the form of an inhaler, is proposed, having the following features.

The liquid dispenser comprises a switchable valve mechanism. It comprises a base, and a discharge head which, for actuation of the valve mechanism, is movable with respect to the base in a superposed axial and rotational movement or in a rotational movement between an opening position, in which the valve mechanism is opened, and a closure position, in which the valve mechanism is closed.

The liquid dispenser is accordingly configured in such a way that, for the purpose of discharge, the user first of all turns the discharge head relative to a base in a first direction of rotation and thereby opens the switching valve. After the discharge has taken place, in particular after the inhalation process has taken place, the user turns the discharge head in the opposite direction and thereby closes the switching valve. The switching valve, which can be opened and closed by rotation movement of the discharge head, is provided as a single liquid valve which on its own permits or suppresses the discharge of liquid. Therefore, besides the rotation movement, no further activation is needed to start the discharge.

In a delivery state of the liquid dispenser, a rotational movement of the discharge head with respect to the base is blocked by a tamper-evident portion that is separable from the dispenser.

In a liquid dispenser according to the invention, provision is therefore made that this liquid dispenser has a tamper-evident portion, i.e. a portion which is to be removed prior to the first use of the dispenser and which prevents a rotational movement of the discharge head relative to the base. It is only when the tamper-evident portion has been removed from the dispenser that the rotational mobility of the discharge head relative to the base is obtained.

Therefore, the tamper-evident portion is preferably a tamper-evident portion which is accessible from the outside in the delivery state of the liquid dispenser and which can be subjected directly to force for the purpose of separation from the rest of the liquid dispenser.

In a configuration of a liquid dispenser according to the invention with a purely rotational mobility of the discharge head relative to the base, the tamper-evident portion must directly prevent the rotation movement. For this purpose, for example, it can be integrally formed both on the discharge head and on the base, such that it has to be separated at least from one of these elements in order for the rotational mobility to be obtained. Alternatively, the tamper-evident portion can also be integrally secured only on the discharge head or only on the base and can be in form-fit engagement with the respective other element before being separated from the liquid dispenser.

Of particular advantage is a configuration in which the discharge head is movable in a superposed axial and rotational movement. In such a configuration, the nature of the tamper-evident portion can be such that it directly suppresses the axial movement of the discharge head relative to the base and in this way also indirectly prevents the rotational movement. This therefore provides an advantageously simple way of ensuring that an axial movement component is also required for opening the valve.

The axial component of the relative mobility is preferably the one which directly opens or closes the valve mechanism through axial movement of the discharge head. However, configurations are also conceivable in which the opening position and the closure position do not require any axial relative movement at the transfer from one position to the other position, but a relative movement between discharge head and base in the axial direction is required for starting up the dispenser.

A tamper-evident portion which directly suppresses the axial movement can in particular be provided as a completely separate component which in the delivery state, for example, is arranged between the discharge head and the base in such a way that these cannot be brought any closer to each other. A separate annular tamper-evident portion of this kind can be fitted in place during assembly and can be removed in its entirety when the dispenser is put to use, without having to be materially separated from one of the components of the discharge head or of the base. It is of advantage, however, if the discharge head has a housing on which the tamper-evident portion is integrally formed. The connection between this housing and the tamper-evident portion can be, for example, in the form of thin bridges that break when required or else in the form of a continuous connecting portion that is configured along its entire length as a thin predetermined breaking point.

Preferably, the discharge head is also at least axially movable between the closure position and the opening position. In the delivery state, the tamper-evident portion blocks the discharge head in the closure position in which the discharge head is arranged in an end position at a maximum distance from the base.

In a liquid dispenser according to the invention, the discharge head and the base are usually guided slidably on each other and thus bear at all times on each other. The aforementioned maximum distance is understood as meaning that the discharge head is in a position relative to the base in which it is pushed to the least possible extent onto the base or into the base. In a dispenser according to the invention, this end position at a maximum distance preferably entails that the valve mechanism is closed. The tamper-evident portion secures this maximum distance and thus indirectly prevents opening of the valve mechanism.

The tamper-evident portion preferably has a cover portion which, before the separation, covers a viewing window of the liquid dispenser, wherein a marking is applied behind the viewing window.

According to this embodiment, the tamper-evident portion thus assumes a dual function. On the one hand, it prevents a relative movement between the discharge head and the base, in the manner already described. On the other hand, with the cover portion, it makes available a portion by means of which a viewing window of the liquid dispenser is closed. When the tamper-evident portion is separated from the dispenser upon the first use, the viewing window of the liquid dispenser is thus also opened, and it can therefore be seen at a glance that the tamper-evident portion has already been removed.

Although the invention focuses principally on liquid dispensers in which there is at least one rotational component of relative mobility in the movement of the discharge head with respect to the base, said aspect of the viewing window covered by the tamper-evident portion in the delivery state is also deemed advantageous in liquid dispensers with a purely axial relative mobility. The same applies to the aspects mentioned subsequently in the context of the viewing window.

In the simplest case, the marking that the user is able to read when the viewing window is opened can be a marking indicating the initial use, for example in the form of the word “OPEN”. In the context of the invention, a marking is to be understood as a marking in the form of actual letters, but also as pictograms or surface areas of contrasting colors that can be equally indicative to the user.

The viewing window is preferably provided in a first housing part which is movable relative to a second housing part, wherein a surface portion of the second housing part is visible through the viewing window. The marking is applied to the second housing part.

The application of the marking to another housing part, not the one in which the viewing window is provided, simplifies matters in terms of production technology. Moreover, on account of the mobility of the two housing parts relative to each other, the marking is not visible all the time, but instead only at certain relative positions, such that a marking that indicates the relative position itself can also be obtained. This applies in particular to embodiments in which two or more markings are applied to the second housing part, such that, on account of the relative mobility, the viewing window in the first housing part shows one marking or the other depending on the operating state of the liquid dispenser. For example, suitable markings could be “ON” and “OFF”.

The cover portion preferably forms a grip portion of the tamper-evident portion and for this purpose has a greater material thickness than the mean material thickness of the tamper-evident portion and/or has a radially outward, exposed arrangement relative to other partial portions of the tamper-evident portion.

This grip portion preferably forms an end of the tamper-evident portion which, particularly preferably, is more or less band-like. Since a grip portion of this kind is preferably larger than the other portions of the tamper-evident portion anyway, it is able to cover a sufficiently large viewing window on the liquid dispenser in the delivery state.

As a result of the thicker material and/or the fact that the grip portion forms the part of the tamper-evident portion located farthest to the outside with respect to the rotation axis, the grip portion is made easier to grasp. Preferably, special material bridges are provided between the housing and the grip portion in order to hold this in position until the user removes the tamper-evident portion.

The stated mobility of the discharge head and of the base relative to each other are preferably formed by at least one slotted guide track on the base or on the discharge head, and by a web or a corresponding cam engaging therein in the manner of a thread. Several slotted guide tracks and correspondingly several webs or cams are preferably provided in order to ensure particularly secure guiding.

It is also of advantage if several markings are provided corresponding to such a configuration with several slotted guide tracks. By virtue of the several slotted guide tracks, the housing parts can be coupled in several positions in the course of assembly. The provision of multiple markings ensures that one of the markings is arranged in the region of the viewing window irrespective of the specific relative position of the parts during the coupling.

The liquid dispenser usually comprises a liquid reservoir which, in view of the intended portable nature of the dispenser, is preferably configured to receive not more than 500 ml, particularly preferably not more than 250 ml. In particular, the liquid reservoir is configured as a pressure accumulator, i.e. for storing the liquid at an overpressure, wherein the overpressure is provided by compressed gas, in particular air, or by an enclosed propellant. A configuration with an accumulator piston that is subjected to force by a spring is also possible.

The liquid reservoir can in particular be that of a liquid dispenser designed in particular as an inhaler and can be filled with one of the following liquids: A saline aqueous solution or an aqueous solution in the form of a Ringer's solution or a buffered solution or an aqueous solution with at least one of the additives carbohydrates, essential oils, menthol and plant extracts, or an aqueous solution containing vitamins, trace elements, manganese or zinc, or an aqueous solution containing at least one of the additives from the group comprising cinnamon oil, tea tree oil, sage oil, thyme oil and lemon balm oil.

The liquid dispenser is designed as an inhaler and has a respiration piece, which is configured as a mouthpiece or as a breathing mask.

### BRIEF DESCRIPTION OF THE DRAWINGS

Further advantages and aspects of the invention will become clear from the claims and from the following description of a preferred illustrative embodiment of the invention explained below with reference to the figures.

FIG. 1 and FIG. 2 show a liquid dispenser according to the invention in an overall view, wherein the liquid reservoir is shown in section in FIG. 2.

FIG. 3 shows a housing part of the base and also a housing part of the discharge head, together with a tamper-evident portion applied to the discharge head.

FIG. 4 shows the housing parts from FIG. 3 in their relative position in the delivery state.

FIGS. 5 and 6 show the two relative end positions of the housing parts from FIGS. 3 and 4 after removal of the tamper-evident portion.

FIG. 7 shows an exploded view of an alternative configuration corresponding to FIG. 3.

### DETAILED DESCRIPTION OF THE ILLUSTRATIVE EMBODIMENTS

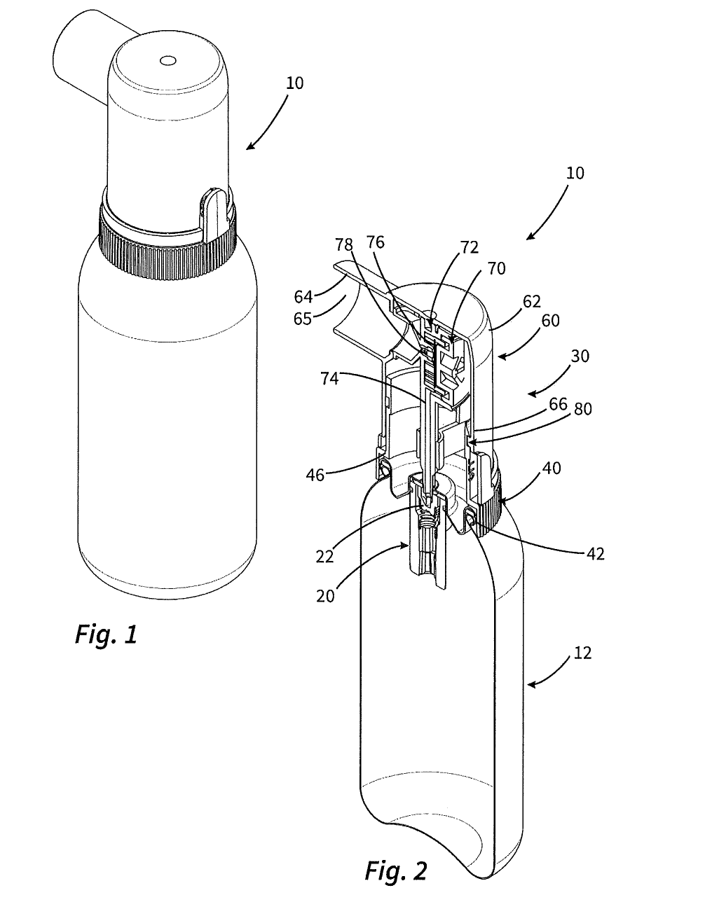

FIGS. 1 and 2 show an embodiment of a dispenser according to the invention in an overall view and in a sectional view. The dispenser 10 according to the invention has a liquid reservoir 12, configured as a pressure accumulator, and also a discharge head 60. The discharge head 60 comprises a base 40, which is connected to the liquid reservoir 12 by means of a snap-fit connection 42 and is non-rotatable with respect to the liquid reservoir 12.

The dispenser moreover comprises a discharge head 60 which, in a manner explained in more detail below, is movable relative to the base 40 in order thereby to control the discharge of liquid.

The discharge head 60 has an outer housing 62 in which two inner components 70, 72 are secured, wherein the inner part 72 comprises a feed pipe 74 which extends into a valve mechanism 20 provided on the liquid reservoir 12 and bears there on an axially movable valve body 22.

In a movement of the discharge head 60 relative to the base 40, in the course of which movement the feed pipe 74 is moved in the direction of the liquid reservoir, liquid flows from the liquid reservoir through the feed pipe 74 and through a filter unit 76 as far as a nozzle unit 78 which, in a manner not shown, comprises a large number of individual nozzle openings. Here, the liquid is atomized and passes into an outlet channel 65 of a respiration piece 64, which is part of the outer housing 62.

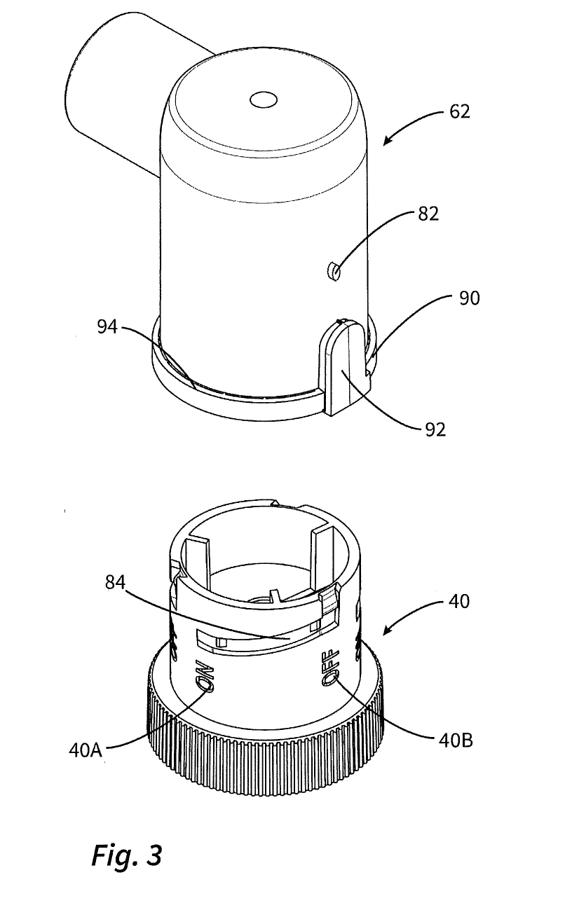

The base 40 and the outer housing 62 of the discharge head 60 together define the mobility of the discharge head 60 relative to the base 40 and thus relative to the liquid reservoir 12. As will be seen from FIG. 3, a slotted guide track 84 is provided in the base component 40. Corresponding to this, a threaded cam 82 is mounted on the inner face of a sleeve portion 66 of the outer housing 62, which threaded cam 82 protrudes into the slotted guide track 84. Since the slotted guide track has the shape of a portion of a helix as shown in FIG. 3, the discharge head 60 is movable relative to the base in a correspondingly superposed relative axial and rotational movement. A rotation movement of the discharge head 60 relative to the base 40 thus brings about indirectly an axial movement, which is suitable for opening and closing the valve mechanism 20.

It will also be seen from FIG. 3 that a tamper-evident portion 90 is provided circumferentially at the lower open end of the outer housing 62. The tamper-evident portion 90 is a tamper-evident band, at the end of which is arranged a grip portion which serves as cover portion 92. The tamper-evident portion 90 along with the grip portion 92 is materially connected to the outer housing 62 by material bridges.

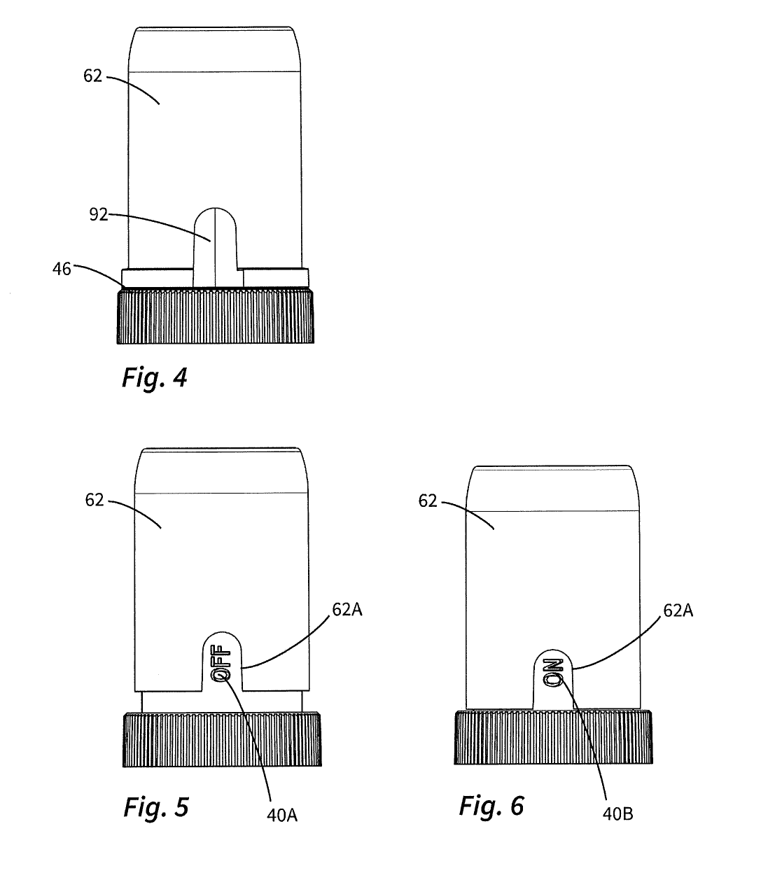

Referring to FIG. 4, it will be seen that an axial movement of the outer housing 62 relative to the base 40 is not possible when the tamper-evident portion 90 is still attached. During such a movement, the tamper-evident portion would collide with a step 46 of the base 40. Since an axial movement is not possible, a superposed rotational and axial movement is also not possible with the tamper-evident portion attached. The tamper-evident portion first has to be removed for the state according to FIG. 5 to arise.

When this has been done, the superposed axial and rotational movement provided for the activation of the dispenser can then take place, and a discharge of liquid can thus be effected.

As FIGS. 5 and 6 show, a viewing window 62A is opened by removal of the tamper-evident portion 90, and in particular of the cover portion 92, which viewing window 62A makes an underlying marking visible. As will be seen from FIG. 3, two such markings are provided on the outer face of the base 40, such that, depending on the relative position of the outer housing 62, it can immediately be seen from the window whether the dispenser is activated or deactivated.

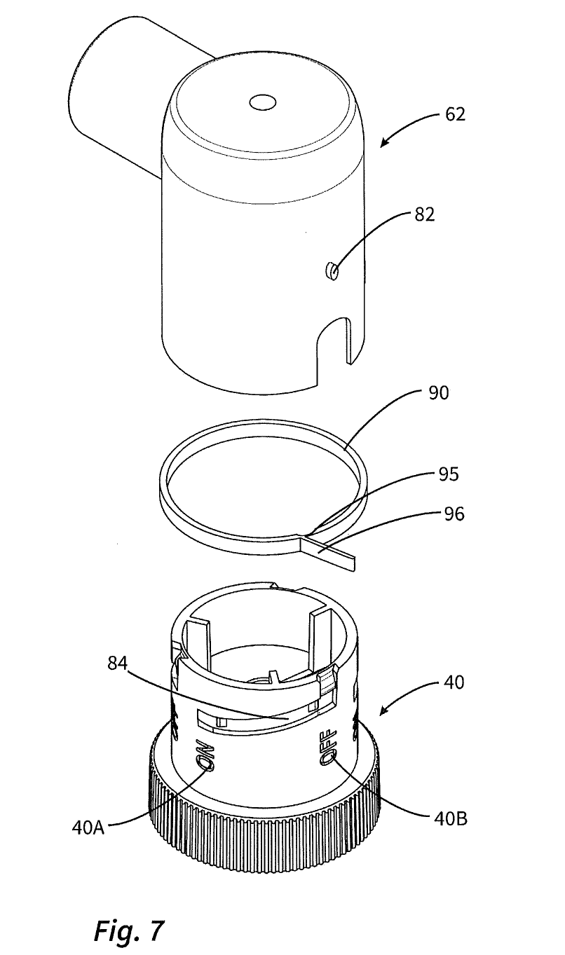

FIG. 7 shows an alternative configuration. Here, the tamper-evident portion 90 is designed as a separate ring. In contrast to the configuration in FIG. 3, the material bridges 94 are thus absent. Instead, the ring in the configuration according to FIG. 7 is closed, and the ring tapers in the region of a predetermined breaking point 95 such that, after a grip tab 96 has been taken hold of and pulled, the ring is opened in the region of the predetermined breaking point and is then able to be removed. Only then can a discharge be effected by rotation of the housing part 62.

The ring shown has an internal diameter greater than the adjacent external diameter of the housing part 62, such that the housing part 62 cannot be moved into the ring.

1. A liquid dispenser for discharging cosmetic or pharmaceutical liquids, in particular in the form of an inhaler, having the following features:
a. the liquid dispenser comprises a switchable valve mechanism, which is opened in order to allow a discharge of liquid and which is closed in order to terminate the discharge of liquid, and
b. the liquid dispenser comprises a base, and
c. the liquid dispenser comprises a discharge head which, for actuation of the valve mechanism, is movable with respect to the base in a superposed axial and rotational movement or in a rotational movement between an opening position, in which the valve mechanism is opened, and a closure position, in which the valve mechanism is closed,
wherein:
d. in a delivery state of the liquid dispenser, a rotational movement of the discharge head with respect to the base is blocked by a tamper-evident portion that is separable from the liquid dispenser.

2. The liquid dispenser as claimed in claim 1, having the following features:
a. the tamper-evident portion is designed as a tamper-evident portion which is accessible from the outside in the delivery state of the liquid dispenser and which can be subjected directly to force for the purpose of separation from the rest of the liquid dispenser.

3. The liquid dispenser as claimed in claim 1, having the following features:
a. the discharge head is movable in a superposed axial and rotational movement, and,
b. in the delivery state of the liquid dispenser, the tamper-evident portion separable from the liquid dispenser blocks the axial movement of the discharge head with respect to the base and thereby indirectly blocks the rotational movement.

4. The liquid dispenser as claimed in claim 1, having the following features:
a. the discharge head has a housing part, and
b. the separable tamper-evident portion is a portion connected integrally to a portion of the housing part.

5. The liquid dispenser as claimed in claim 1, having the following feature:
a. the separable tamper-evident portion is designed as a separate component having a predetermine breaking point, which has to be destroyed for the purpose of separating the tamper-evident portion.

6. The liquid dispenser as claimed in claim 1, having the following feature:
a. the separable tamper-evident portion is an annular portion which surrounds the liquid dispenser by at least 180°, preferably by at least 300°, in particular preferably completely.

7. The liquid dispenser as claimed in claim 1, having the following features:
a. the discharge head is axially movable between the closure position and the opening position, and,
b. in the delivery state, the tamper-evident portion blocks the discharge head in the closure position, in which the discharge head is arranged in an end position at a maximum distance from the base.

8. The liquid dispenser as claimed in claim 1, having the following feature:
a. the tamper-evident portion has a cover portion which, before the separation, covers a viewing window of the liquid dispenser, wherein a marking is applied behind the viewing window.

9. The liquid dispenser as claimed in claim 8, having the following features:
a. the viewing window is provided in a first housing part which is movable relative to a second housing part, wherein a surface portion of the second housing part is visible through the viewing window, and
b. the marking is applied to the second housing part.

10. The liquid dispenser as claimed in claim 9, having the following feature:
a. at least two markings are applied to the second housing part and, depending on the position of the first housing part relative to the second housing part, are visible through the viewing window or are concealed by the first housing part.

11. The liquid dispenser as claimed in claim 8, having the following feature:
a. the cover portion forms a grip portion of the tamper-evident portion and for this purpose has a greater material thickness than the mean material thickness of the tamper-evident portion and/or has a radially outward, exposed arrangement relative to the other partial portions of the tamper-evident portion.

12. The liquid dispenser as claimed in claim 1, having at least one of the following features:
a. the base and the discharge head are movable relative to each other in a superposed axial and rotational movement by means of a slotted guide track in which a slotted guide slider is arranged, and/or
b. the liquid dispenser comprises a liquid reservoir for storing liquid prior to discharge, which liquid reservoir is preferably configured as a pressure accumulator, and/or
c. the liquid reservoir is configured to receive a maximum of 500 ml, preferably a maximum of 250 ml, and/or
d. the liquid reservoir is filled with:
a saline aqueous solution or
an aqueous solution in the form of a Ringer's solution or a buffered solution or an aqueous solution with at least one of the additives carbohydrates, essential oils, menthol and plant extracts or
an aqueous solution containing vitamins, trace elements, manganese or zinc, or
an aqueous solution with at least one of the additives from the group comprising cinnamon oil, tea tree oil, sage oil, thyme oil, lemon balm, and/or
e. the liquid dispenser is configured as an inhaler and has a respiration piece, which is configured as a mouthpiece or as a breathing mask, and/or
f. the respiration piece has a channel portion which is oriented in a direction deviating from an actuation direction of the discharge head and which preferably encloses with the latter an angle of between 45° and 135°, in particular preferably an angle of between 70° and 110°.
